(19) United States
(12) Patent Application Publication
(10) Pub. No.: US 2021/0000430 A1
(43) Pub. Date: Jan. 7, 2021

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventors: Takeshi FUKUDA, Kanagawa (JP); Haruyasu NAKATSUGAWA, Kanagawa (JP)

(73) Assignee: FUJIFILM Corporation, Tokyo (JP)

(21) Appl. No.: 16/920,263

(22) Filed: Jul. 2, 2020

(30) Foreign Application Priority Data
Jul. 5, 2019 (JP) 2019-126030

(51) Int. Cl. A61B 5/00; G16H 10/60; G16H 15/00; G16H 50/70; G16H 50/50; G16H 70/60; G16H 50/20; A61D 99/00

(52) U.S. Cl. CPC A61B 5/742; G16H 10/60; G16H 15/00; G16H 50/70; A61B 2503/40; G16H 70/60; G16H 50/20; A61B 5/7475; A61D 99/00; G16H 50/50

(57) ABSTRACT

Provided are an information processing apparatus, an information processing method, and an information processing program capable of supporting an effective diagnosis.

The information processing apparatus receives an input of a symptom of a subject, presents a candidate for a cause of the symptom based on the received symptom, receives the cause of the symptom input by a user among the presented candidates for the cause of the symptom, and presents a candidate for a disease based on the received cause of the symptom.

### CROSS-REFERENCE TO RELATED APPLICATIONS

This application is based on and claims priority under 35 USC 119 from Japanese Patent Application No. 2019-126030 filed on Jul. 5, 2019, the disclosure of which is incorporated herein by reference in its entirety.

### BACKGROUND OF THE INVENTION

### 1. Field of the Invention

The present disclosure relates to an information processing apparatus, an information processing method, and a computer-readable storage medium storing an information processing program.

### 2. Description of the Related Art

An electronic medical chart system that presents a candidate for a suspicious disease based on a symptom input to an electronic medical chart has been disclosed (see JP2018-081527A).

An inquiry system that presents candidates for a disease in descending order of the probability of being suspected of a disease based on an answer to a question in an inquiry for confirmation of symptoms has been disclosed (see JP6496876B).

For the treatment of a disease in a subject, not only the disease causing the symptoms but also the cause of the disease is important information. This is because, for example, even for the same disease, in a case where the cause of the disease is different, the treatment policy may be different, such as different drugs to be administered. Therefore, specifying the cause of the disease in addition to the disease is preferable for a user such as a doctor to perform an effective diagnosis. However, in JP2018-081527A and JP6496876B, it is only considered to present the candidate for the disease based on the symptom.

### SUMMARY OF THE INVENTION

The present disclosure has been made in view of the above circumstances, and provides an information processing apparatus, an information processing method, and a computer-readable storage medium storing an information processing program capable of supporting an effective diagnosis.

In order to achieve the above object, an information processing apparatus according to the present disclosure includes a processor that is configured to receive, via an input device, an input of a symptom of a subject, and present, via a display, a candidate for a cause of the symptom based on the symptom received.

A computer-readable storage medium storing an information processing program according to the present disclosure causes a computer to execute receiving, via an input device, an input of a symptom of a subject; and presenting, via a display, a candidate for a cause of the symptom based on the received symptom.

The information processing apparatus according to the present disclosure include a computer-readable storage medium that stores a command for the computer to execute, and a processor configured to execute the stored command, in which the processor receives, via an input device, an input of the symptom of the subject, and presents, via a display, the candidate for the cause of the symptom based on the received symptom.

According to the present disclosure, it is possible to support an effective diagnosis.

### BRIEF DESCRIPTION OF THE DRAWINGS

FIG. 1 is a block diagram showing an example of a configuration of an information processing system.

FIG. 2 is a block diagram showing an example of a hardware configuration of an information processing apparatus.

FIG. 3 is a diagram for explaining a differential diagnosis DB.

FIG. 4 is a diagram for explaining a differential diagnosis DB in a case where a subject is a dog.

FIG. 5 is a block diagram showing an example of a functional configuration of the information processing apparatus.

FIG. 6 is a diagram showing an example of a presentation screen of a candidate for a symptom.

FIG. 7 is a diagram showing an example of a presentation screen of a candidate for a cause.

FIG. 8 is a diagram showing an example of a presentation screen of a candidate for a disease.

FIG. 9 is a diagram showing an example of a presentation screen of a treatment policy.

FIG. 10 is a flowchart showing an example of presentation processing.

FIG. 11 is a diagram for explaining an applicable degree of the symptom.

FIG. 12 is a diagram for explaining relevancy between the symptom and the cause.

FIG. 13 is a diagram for explaining a combination of the applicable degree of the symptom and the relevancy between the symptom and the cause.

### DESCRIPTION OF THE PREFERRED EMBODIMENTS

Hereinafter, embodiments of the technology of the present disclosure will be described in detail with reference to the drawings.

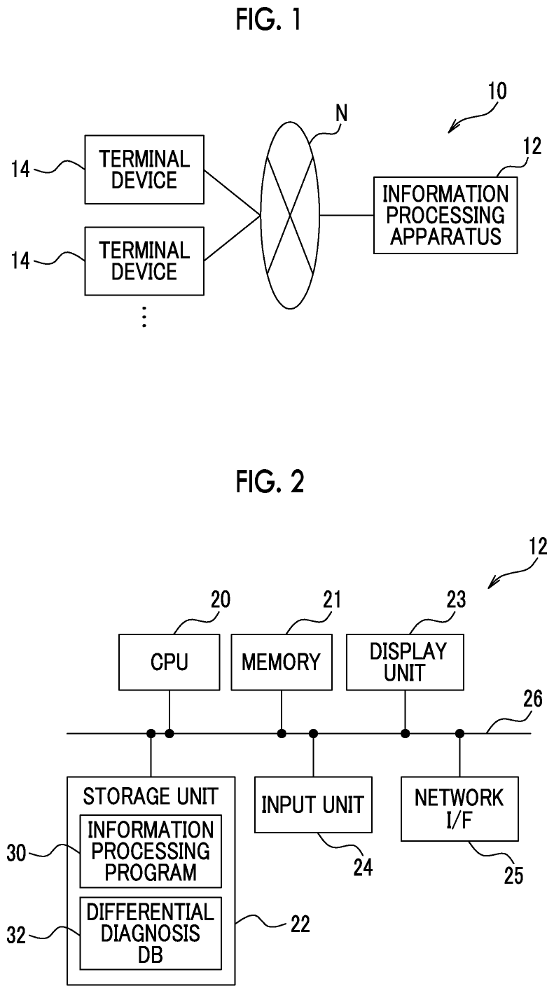

A configuration of an information processing system 10 according to the present embodiment will be described with reference to FIG. 1. As shown in FIG. 1, the information processing system 10 includes an information processing apparatus 12 and a plurality of terminal devices 14. The information processing apparatus 12 and the plurality of terminal devices 14 are connected to a network N, and can communicate with each other via the network N.

The information processing apparatus 12 is installed in, for example, an animal hospital. Examples of the information processing apparatus 12 include a server computer. The information processing apparatus 12 may be a cloud server. For example, the terminal device 14 is installed in the animal hospital, and is used by a user such as a veterinarian. Examples of the terminal device 14 include a personal computer and a tablet computer. In the present specification, “animal” means an animal, such as a dog or a cat, excluding “human”.

A hardware configuration of the information processing apparatus 12 according to the present embodiment will be described with reference to FIG. 2. As shown in FIG. 2, the information processing apparatus 12 includes a central processing unit (CPU) 20, a memory 21 as a temporary storage area, and a nonvolatile storage unit 22. Also, the information processing apparatus 12 includes a display unit 23 such as a liquid crystal display, an input unit 24 such as a keyboard and a mouse, and a network interface (I/F) 25 connected to the network N. The CPU 20, the memory 21, the storage unit 22, the display unit 23, the input unit 24, and the network I/F 25 are connected to a bus 26.

The storage unit 22 is realized by a hard disk drive (HDD), a solid state drive (SSD), or a flash memory. The storage unit 22 as a storage medium stores information processing program 30. The CPU 20 reads the information processing program 30 from the storage unit 22, expands the information processing program 30 in the memory 21, and executes the expanded information processing program 30. The storage unit 22 stores a differential diagnosis database (DB) 32 used for a differential diagnosis performed to narrow down a disease causing the symptom.

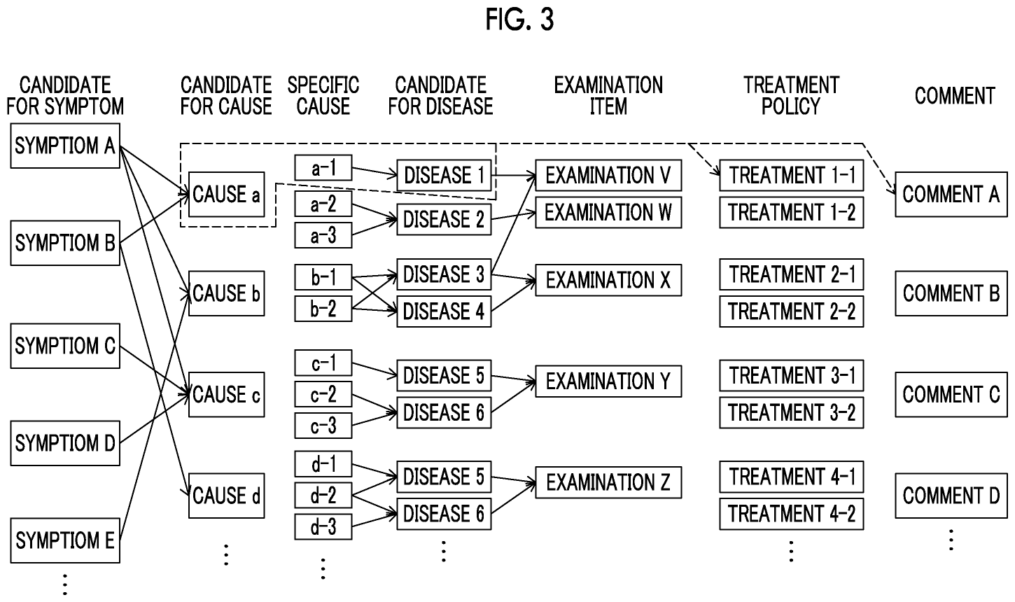

The differential diagnosis DB 32 according to the present embodiment will be described with reference to FIG. 3. As shown in FIG. 3, a candidate for a cause is stored in association with each symptom listed as a candidate for the symptom in the differential diagnosis DB 32. In an example of FIG. 3, “cause a” “cause b”, and “cause c” are associated with “symptom A”. The symptom includes a symptom input by the user using the free input field in addition to a symptom presented to the user as an option.

Also, more specific cause is stored in association with each cause listed as the candidate for the cause in the differential diagnosis DB 32. In an example of FIG. 3, “a-1” “a-2”, and “a-3” as more specific causes are associated with “cause a”. The candidate for the disease is stored in association with each combination of the cause listed as the candidate for the cause and the specific cause thereof in the differential diagnosis DB 32. In an example of FIG. 3, “disease 1” is associated with the combination of “cause a” and “cause a-1”. The candidate for the disease may be associated with only the cause listed as the candidate for the cause, and the candidate for the disease may be associated with only the specific cause. The cause of the symptom may be one layer or three or more layers, instead of two layers of the cause candidate and the specific cause.

An examination item, a treatment policy, and a comment are associated with each combination of the cause listed as the candidate for the cause, the specific cause, and the disease listed as the candidate for the disease in the differential diagnosis DB. In an example of FIG. 3, “examination V”, “treatment 1-1”, and “comment A” are associated with a combination of “cause a”, “cause a-1”, and “disease 1”.

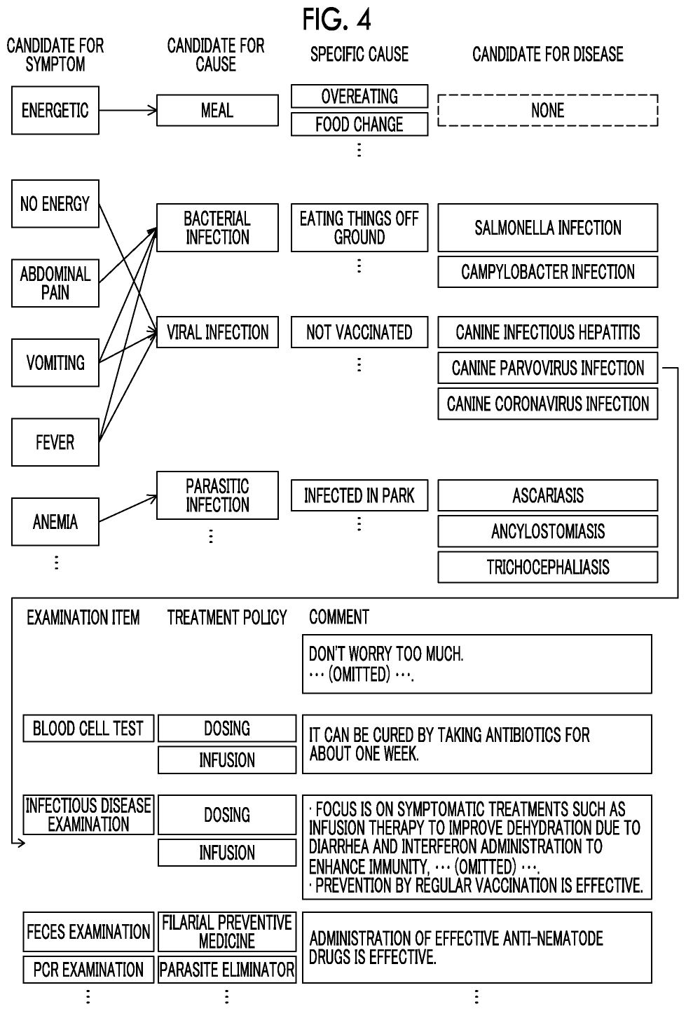

As a more specific example, FIG. 4 shows an example of the differential diagnosis DB 32 in a case where the subject animal is a dog and the main symptom is “diarrhea”. In this case, the candidate for the cause (“meal” and “bacterial infection” in an example of FIG. 4) is associated with a candidate for the specific symptom of diarrhea (“energetic” and “abdominal pain” in an example of FIG. 4) as shown in FIG. 4. In the example of FIG. 4, the cause of “meal” is associated with the symptom of “energetic”. The causes of “bacterial infection” and “viral infection” are associated with the symptom of “vomiting”.

The specific cause (“overeating” and “not vaccinated” in the example of FIG. 4) is associated with the candidate for the cause. In the example of FIG. 4, the more specific causes of “overeating” and “food change” are associated with the candidate for the cause of “meal”. The candidate for the disease (“salmonella infection” in the example of FIG. 4) is associated with the combination of the candidate for the cause and the specific cause. In the example of FIG. 4, the diseases of “salmonella infection” and “campylobacter infection” are associated with the combination of “bacterial infection” and “eating things off the ground”.

The examination item and the treatment policy are associated with the combination of the candidate for the cause, the specific cause, and the disease. For example, the examination item of “blood cell test” and the treatment policy “dosing” and “infusion” are associated with the combination of the cause of “bacterial infection”, the specific cause of “eating things off the ground”, and the disease of “salmonella infection”.

The combination of the cause, the specific cause, and the disease is associated with the comment regarding treatment and handling of the combination. The comment is, for example, as written in a veterinary technical book.

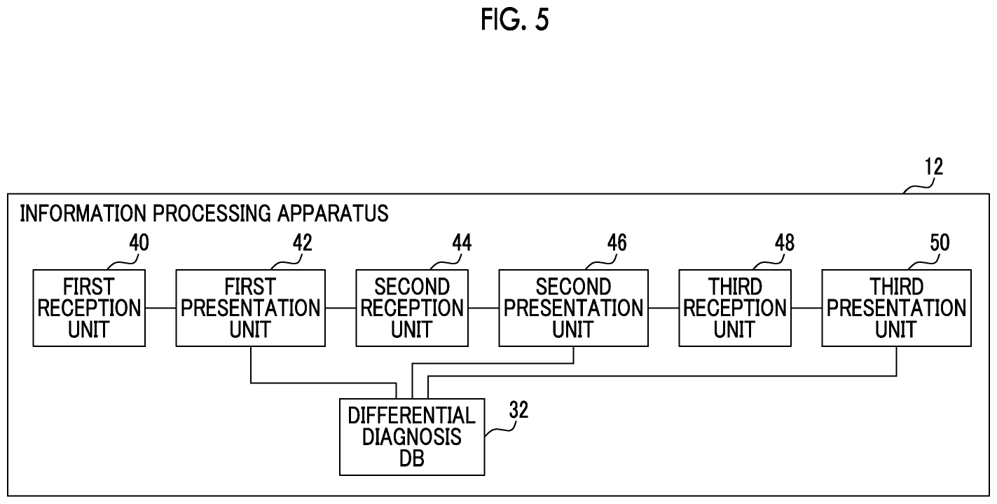

A functional configuration of the information processing apparatus 12 according to the present embodiment will be described with reference to FIG. 5. As shown in FIG. 5, the information processing apparatus 12 includes a first reception unit 40, a first presentation unit 42, a second reception unit 44, a second presentation unit 46, a third reception unit 48, and a third presentation unit 50. The CPU 20 executes the information processing program 30 to function as the first reception unit 40, the first presentation unit 42, the second reception unit 44, the second presentation unit 46, the third reception unit 48, and the third presentation unit 50.

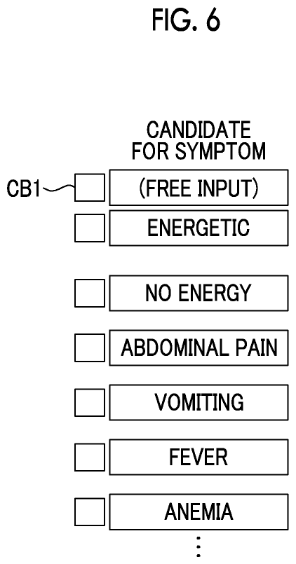

The user such as a veterinarian of the animal hospital inputs the main symptom of the subject via an operation unit of the terminal device 14. In a case where the main symptom is input, a presentation screen for the candidate for the symptom shown in FIG. 6 is displayed on a display unit of the terminal device 14. As shown in FIG. 6, for example, the candidate for the specific symptom associated with the main symptom of “diarrhea”, and a check box CB1 for selecting each candidate for the symptom are displayed on the display unit of the terminal device 14. In the present embodiment, in a case where there is no applicable symptom among the presented candidates for the symptom, the free input field for the user to input a character string of the symptom is also displayed.

The user selects the symptom by designating the check box CB1 corresponding to the applicable symptom among the presented candidates for the symptom or inputs the symptom of the subject by inputting the symptom in the free input field. The first reception unit 40 receives the symptom of the subject input from the terminal device 14.

The first presentation unit 42 presents the candidate for the cause of the symptom based on the symptom received by the first reception unit 40. Specifically, the first presentation unit 42 refers to the differential diagnosis DB 32, and outputs and presents the candidate for the cause corresponding to the symptom received by the first reception unit 40 and the specific cause corresponding to the candidate for the cause, to the terminal device 14.

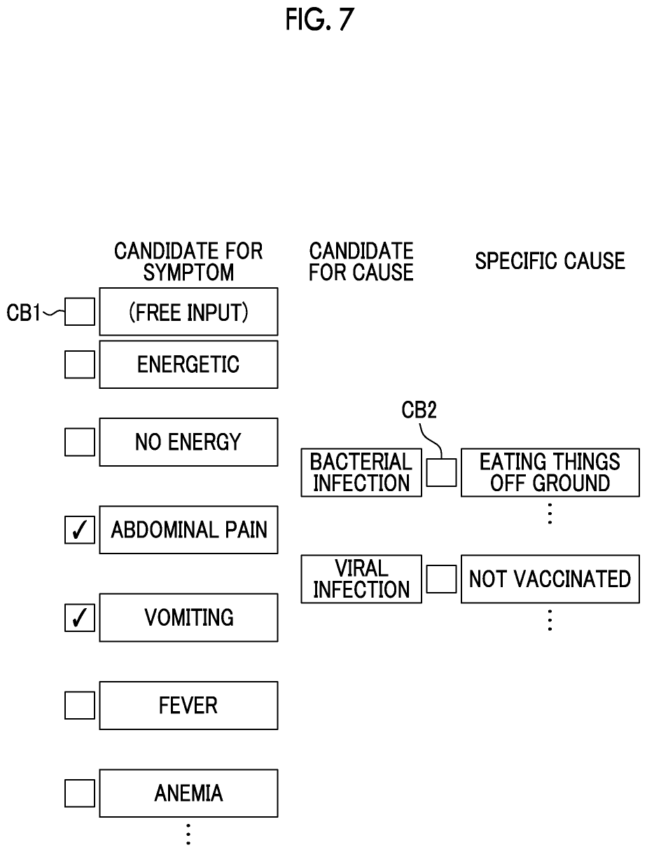

The presentation screen for the candidate for the cause shown in FIG. 7 is displayed on the display unit of the terminal device 14 by the presentation of the first presentation unit 42. As shown in FIG. 7, the candidate for the cause corresponding to the symptom selected by the user and the specific cause, and a check box CB2 for selecting each specific cause are displayed on the display unit of the terminal device 14. In an example of FIG. 7, “abdominal pain” and “vomiting” are selected as the symptom, “bacterial infection” corresponding to “abdominal pain”, and “bacterial infection” and “viral infection” corresponding to “vomiting” are presented. The free input field may be displayed on the presentation screen for the candidate for the cause, similarly to the presentation screen for the candidate for the symptom shown in FIG. 6.

The user selects one of the presented candidates for the cause, which is presumed to be the cause of the symptom, according to the results of the palpation of the subject animal and the inquiry to the owner of the animal. The cause of the symptom is input from the terminal device 14 to the information processing apparatus 12 by the selection of the user. The second reception unit 44 receives an input of the cause of the symptom from the terminal device 14.

The second presentation unit 46 presents the candidate for the disease based on the cause of the symptom received by the second reception unit 44. Specifically, the second presentation unit 46 refers to the differential diagnosis DB 32, and outputs and presents the candidate for the disease associated with the combination of the cause selected by the user, which is received by the second reception unit 44 as the cause of the symptom, and the specific cause, to the terminal device 14.

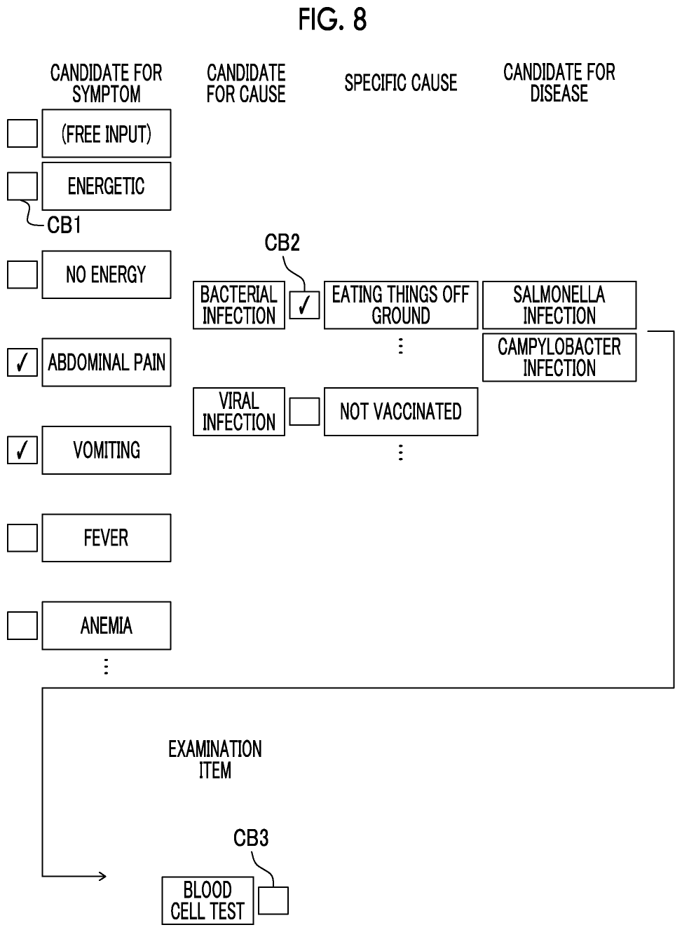

The presentation screen for the candidate for the disease shown in FIG. 8 is displayed on the display unit of the terminal device 14 by the presentation of the second presentation unit 46.

As shown in FIG. 8, the candidate for the disease corresponding to the cause selected by the user is displayed on the display unit of the terminal device 14. In an example of FIG. 8, the combination of the cause of “bacterial infection” as the cause of the symptom and “eating things off the ground” as the more specific cause is selected, and “salmonella infection” and “campylobacter infection” are presented as the candidate for the disease corresponding to the selected cause.

The second presentation unit 46 further presents the candidate for the examination item based on the presented candidate for the disease. Specifically, the second presentation unit 46 refers to the differential diagnosis DB 32, and outputs and presents the examination item corresponding to each combination of the cause received by the second reception unit 44 and the candidate for the disease associated with the cause, to the terminal device 14. In an example of FIG. 8, the combination of the causes of “bacterial infection” and “eating things off the ground” and “salmonella infection”, and the examination item of “blood cell test” associated with the combination of the causes of “bacterial infection” and “eating things off the ground” and “campylobacter infection” are presented. In the example of FIG. 8, a check box CB3 for the user to designate the examination item is also displayed.

The user determines the examination item necessary for specifying the disease, and designates the examination item to be performed. In a case where the examination item is designated, the subject is examined according to the designated examination item. In a case where the examination of the subject ends, the user receives the examination result, and the user specifies the disease of the subject based on the examination result. Then, the user designates the specified disease on the presentation screen for the candidate for the disease. The specified disease is input from the terminal device 14 to the information processing apparatus 12 by the designation of the user. The third reception unit 48 receives an input of the disease from the terminal device 14.

The third presentation unit 50 presents the treatment policy and the comment based on the cause of the symptom received by the second reception unit 44, and the disease received by the third reception unit 48. Specifically, the third presentation unit 50 refers to the differential diagnosis DB 32, and outputs and presents the treatment policy and the comment corresponding to the combination of the cause of the symptom received by the second reception unit 44 and the disease received by the third reception unit 48, to the terminal device 14.

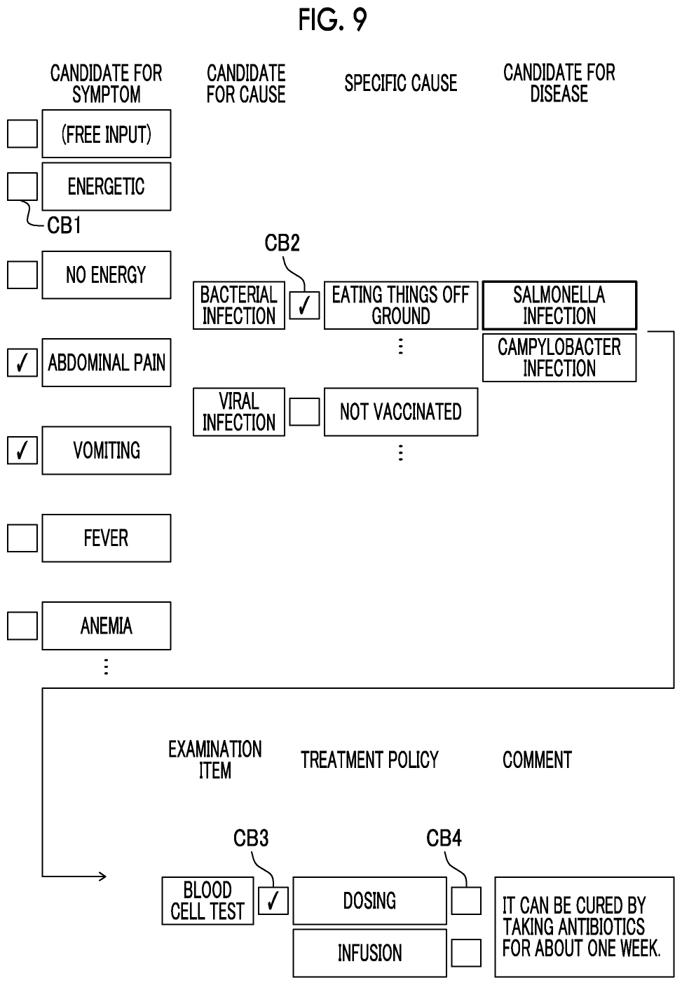

The presentation screen for the treatment policy shown in FIG. 9 is displayed on the display unit of the terminal device 14 by the presentation of the third presentation unit 50. As shown in FIG. 9, the treatment policy and the comment corresponding to the cause selected by the user and the disease specified by the user, and a check box CB4 for selecting the treatment policy are displayed on the display unit of the terminal device 14. In an example of FIG. 9, “blood cell test” is selected as the examination item, as a result of the examination, the disease is specified as “salmonella infection” by the user, “dosing” and “infusion” as the treatment policy are presented, and the comment of “It can be cured by taking antibiotics for about one week.” is presented.

The user decides the treatment policy with reference to the results of the palpation of the subject animal and the inquiry to the owner of the animal and the comments, and selects the decided treatment policy. The cause, the disease, the examination item, the examination result, and the treatment policy which are selected by the above processing are stored in the storage unit 22 as the electronic medical chart of the subject.

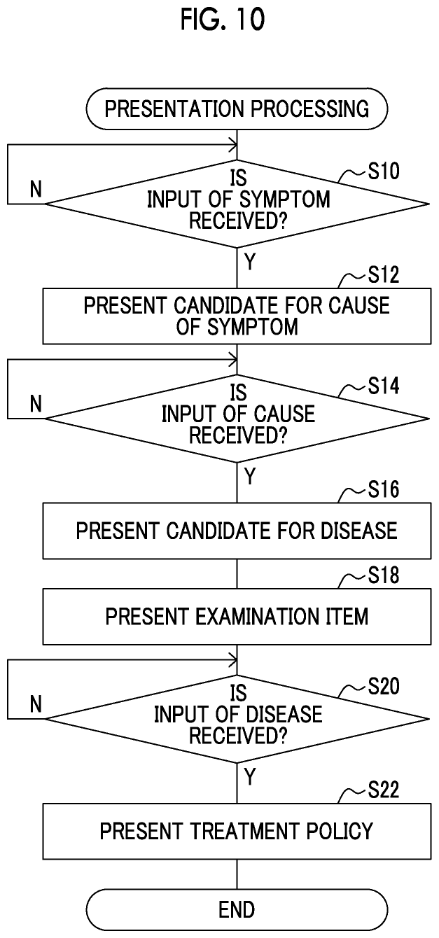

The operation of the information processing apparatus 12 according to the present embodiment will be described with reference to FIG. 10. The CPU 20 executes the information processing program 30 to execute presentation processing shown in FIG. 10.

In step S10 of FIG. 10, the first reception unit 40 waits until the first reception unit 40 receives an input of the symptom of the subject from the terminal device 14. In a case where the first reception unit 40 receives an input of the symptom of the subject, the determination in step S10 is affirmative, and the processing proceeds to step S12. In step S12, as described above, the first presentation unit 42 refers to the differential diagnosis DB 32, and presents the candidate for the cause corresponding to the symptom received in the processing of step S10 and the specific cause corresponding to the candidate for the cause.

In step S14, the second reception unit 44 receives an input of the cause of the symptom from the terminal device 14. In a case where the user inputs the cause of the symptom according to the presentation in step S12 and the second reception unit 44 receives an input of the cause of the symptom, the determination in step S14 is affirmative, and the processing proceeds to step S16.

In step S16, as described above, the second presentation unit 46 refers to the differential diagnosis DB 32, and presents the candidate for the disease associated with the combination of the cause which is received in the processing of step S14 as the cause of the symptom, and the specific cause. In step S18, as described above, the second presentation unit 46 refers to the differential diagnosis DB 32, and presents the examination item corresponding to each combination of the cause received in the processing of step S14 and the candidate for the disease associated with the cause.

In step S20, the third reception unit 48 receives an input of the disease from the terminal device 14. As a result of the examination performed according to the presentation in step S18, the disease is input by the user, and in a case where the third reception unit 48 receives an input of the disease, the determination in step S20 is affirmative, and thus the processing proceeds to step S22.

In step S22, as described above, the third presentation unit 50 refers to the differential diagnosis DB 32, and presents the treatment policy and the comment corresponding to the combination of the cause of the symptom received in the processing of step S144 and the disease received in the processing of step S20. In a case where the processing of step S22 ends, the presentation processing ends.

As described above, according to the present embodiment, the candidate for the disease is not directly presented from the symptom, the candidate for the cause is presented from the symptom and the candidate for the disease corresponding to the selected cause is presented, and thus the treatment policy can be decided according to the combination of the cause and the disease. Therefore, it is possible to support an effective diagnosis.

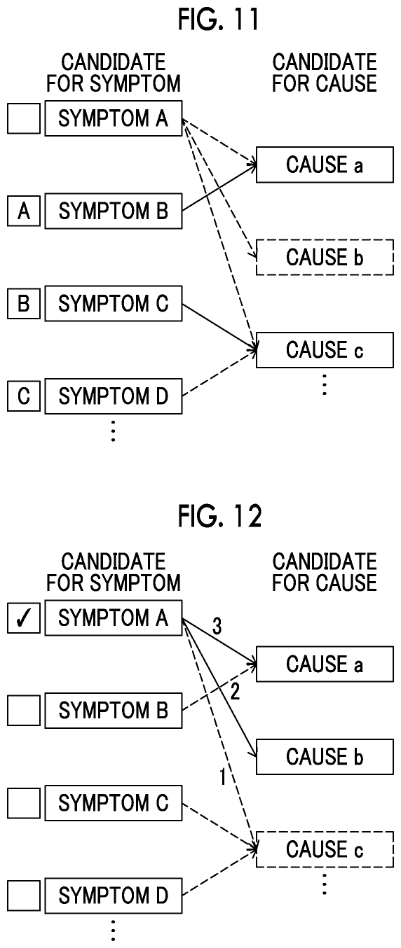

In a case where a plurality of symptoms of the subject is input on the presentation screen (see FIG. 6) for the candidate for the symptom according to the above embodiment, the applicable degree of each symptom may be input. For example, as shown in FIG. 11, four applicable degrees are input as follows: a fairly applicable symptom (“A” in the example of FIG. 11), a slightly applicable symptom (“B” in the example of FIG. 11), a symptom that is neither (no mark in the example of FIG. 11), and a not applicable symptom (“C” in the example of FIG. 11).

In this case, the first reception unit 40 further receives the applicable degree of each symptom, and the first presentation unit 42 presents the candidate for the cause of the symptom according to the priority based on the applicable degree received by the first reception unit 40. Specifically, for example, the first presentation unit 42 imparts a larger value as the applicable degree becomes larger such as “2” for “A”, “1” for “B”, “0” for no mark, and “−2” for “C”, and presents the candidate for the cause associated with the symptom having the value equal to or more than a threshold value. In an example of FIG. 11, the candidate for the cause associated with the symptom having the applicable degree equal to or more than “1” is presented. In the example, the first presentation unit 42 may present the candidate for the cause associated with the symptom in the descending order of the value indicating the applicable degree of the symptom, or may highlight and display the candidate for the cause associated with the symptom having the maximum value. Also, the first presentation unit 42 may set the applicable degree of the symptom input in the free input field to be higher than the applicable degree of the symptom displayed as an option.

In the differential diagnosis DB 32 according to the above embodiment, the relevancy indicating a degree of relevance may be set between the symptom of the subject and the candidate for the cause associated with the symptom. For example, as shown in FIG. 12, a higher value is set as the relevancy as the relevance between the symptom and the candidate is high. In this case, for example, the first presentation unit 42 presents the candidate for the cause of the symptom according to the priority based on the relevancy. In an example of FIG. 12, the candidate for the cause among the candidates for the cause associated with the symptom selected by the user, which has the relevancy equal to or more than the threshold value (2 or more in the example of FIG. 12), is presented. In this case, on the presentation screen for the candidate for the cause, the arrow from the candidate for the symptom associated with the candidate for the cause is displayed and the arrow is made thicker as the relevancy is higher, and thus the relevancy may be visually recognizable.

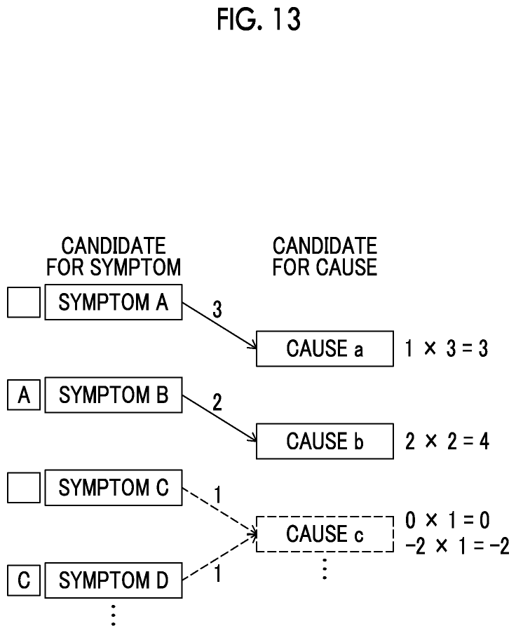

As an example, as shown in FIG. 13, the first presentation unit 42 may present the candidate for the cause of the symptom according to the priority based on the combination of the applicable degree and the relevancy. In an example of FIG. 13, the candidate for the cause is presented in which the value is equal to or more than the threshold value (3 or more in the example of FIG. 13), which is obtained by integrating the value indicating the applicable degree and the value indicating the relevancy.

In the above embodiment, the first presentation unit 42 may present the candidate for the cause of the symptom according to a species of the animal. In this case, an example in which the differential diagnosis DB 32 according to the above embodiment is prepared in advance for each species of the animal such as a dog or a cat. The first presentation unit 42 may present the candidate for the cause of the symptom according to a breed of the animal (for example, a dog breed such as a chihuahua in a case where the animal is a dog).

In the above embodiment, as a hardware structure of processing units such as the first reception unit 40, the first presentation unit 42, the second reception unit 44, the second presentation unit 46, the third reception unit 48, and the third presentation unit 50, which execute various processing, various processors shown below can be used. The various processors include a dedicated electric circuit which is a processor having a circuit configuration specifically designed to execute specific processing such as a programmable logic device (PLD) which is a processor whose circuit configuration can be changed after manufacturing FPGA or an application specific integrated circuit (ASIC), in addition to the CPU which is a general processor that executes a software (the program) and functions as various processing units.

One processing unit may be configured by one of various processors, or may be configured by the combination of two or more processors of the same type or different types (for example, the combination of a plurality of FPGAs or the combination of the CPU and the FPGA). Also, a plurality of processing units may be configured by one processor. As an example of configuring a plurality of processing units by one processor, first, there is an example in which a combination of one or more CPUs and the software configure one processor, and the processor functions as a plurality of processing units, as represented by computers such as a client and a server. Second, there is an example in which the processor that realizes the functions of the entire system including a plurality of processing units with one integrated circuit (IC) chip is used, as represented by the system on chip (SoC). As described above, various processing units are configured by using one or more above processors as the hardware structure.

Further, as a hardware structure of these various processors, more specifically, a circuitry combining circuit elements such as semiconductor elements can be used.

In the above embodiment, the embodiment in which the information processing program 30 is stored (installed) in advance in the storage unit 22, but the embodiment is not limited thereto. The information processing program 30 may be provided as a form recorded on the recording medium such as compact disc read only memory (CD-ROM), digital versatile disc read only memory (DVD-ROM), and universal serial bus (USB) memory. The information processing program 30 may be downloaded from the external device via the network.

The information processing apparatus according to the present disclosure may receive, via the input device, the cause of the symptom input by a user among the candidates for the cause of the symptom presented, and present, via the display, a candidate for a disease based on the cause of the symptom received by the second reception unit.

The information processing apparatus according to the present disclosure may receive, via the input device, the disease input by the user among the candidates for the disease, and present, via the display, a candidate for an examination item based on the disease received.

In the information processing apparatus according to the present disclosure, in a case where a plurality of the symptoms of the subject are input, the processor may further receive, via the input device, an applicable degree of each of the symptoms, and the processor may present, via the display, the candidate for the cause of the symptom in accordance with a priority based on the applicable degree.

In the information processing apparatus according to the present disclosure, relevancy indicating a degree of relevance may be set between the symptom of the subject and the candidate for the cause associated with the symptom, and the processor may present, via a display, the candidate for the cause of the symptom in accordance with a priority based on the relevancy.

In the information processing apparatus according to the present disclosure, the subject may be an animal, and the processor may present the candidate for the cause of the symptom in accordance with a species of the animal.

What is claimed is:

1. An information processing apparatus comprising a processor that is configured to:
receive, via an input device, an input of a symptom of a subject; and
present, via a display, a candidate for a cause of the symptom based on the symptom received.

2. The information processing apparatus according to claim 1,
wherein the processor is further configured to:
receive, via the input device, the cause of the symptom input by a user among the candidates for the cause of the symptom presented; and
present, via the display, a candidate for a disease based on the cause of the symptom received.

3. The information processing apparatus according to claim 2,
wherein the processor is further configured to present, via the display, a candidate for an examination item based on the presented candidate for the disease.

4. The information processing apparatus according to claim 1,
wherein in a case where a plurality of the symptoms of the subject are input, the processor further receives, via the input device, an applicable degree of each of the symptoms, and presents, via the display, the candidate for the cause of the symptom in accordance with a priority based on the applicable degree.

5. The information processing apparatus according to claim 2,
wherein in a case where a plurality of the symptoms of the subject are input, the processor further receives, via the input device, an applicable degree of each of the symptoms, and presents, via the display, the candidate for the cause of the symptom in accordance with a priority based on the applicable degree.

6. The information processing apparatus according to claim 3,
wherein in a case where a plurality of the symptoms of the subject are input, the processor further receives, via the input device, an applicable degree of each of the symptoms, and presents, via the display, the candidate for the cause of the symptom in accordance with a priority based on the applicable degree.

7. The information processing apparatus according to claim 1,
wherein relevancy indicating a degree of relevance is set between the symptom of the subject and the candidate for the cause associated with the symptom, and the processor presents, via the display, the candidate for the cause of the symptom in accordance with a priority based on the relevancy.

8. The information processing apparatus according to claim 2,
wherein relevancy indicating a degree of relevance is set between the symptom of the subject and the candidate for the cause associated with the symptom, and the processor presents, via the display, the candidate for the cause of the symptom in accordance with a priority based on the relevancy.

9. The information processing apparatus according to claim 3,
wherein relevancy indicating a degree of relevance is set between the symptom of the subject and the candidate for the cause associated with the symptom, and the processor presents, via the display, the candidate for the cause of the symptom in accordance with a priority based on the relevancy.

10. The information processing apparatus according to claim 1,
wherein the subject is an animal, and the processor presents, via the display, the candidate for the cause of the symptom in accordance with a species of the animal.

11. The information processing apparatus according to claim 2,
wherein the subject is an animal, and the processor presents, via the display, the candidate for the cause of the symptom in accordance with a species of the animal.

12. The information processing apparatus according to claim 3,
wherein the subject is an animal, and the processor presents, via the display, the candidate for the cause of the symptom in accordance with a species of the animal.

13. An information processing method comprising:
by a computer,
executing processing of receiving, via an input device, an input of a symptom of a subject; and
presenting, via a display, a candidate for a cause of the symptom based on the received symptom.

14. A non-transitory computer-readable storage medium storing an information processing program causing a computer to execute:
receiving, via an input device, an input of a symptom of a subject; and
presenting, via a display, a candidate for a cause of the symptom based on the received symptom.
